(19) United States
(12) Patent Application Publication
(10) Pub. No.: US 2016/0331796 A1
(43) Pub. Date: Nov. 17, 2016

(54) ENRICHED AMARANTHUS BLITOIDES COMPOSITIONS AND USES THEREOF

(71) Applicant: David AUDINO, Park Ridge, IL (US)

(72) Inventor: David AUDINO, Park Ridge, IL (US)

(21) Appl. No.: 15/220,846

(22) Filed: Jul. 27, 2016

Related U.S. Application Data
(63) Continuation of application No. 12/976,524, filed on Dec. 22, 2010, now abandoned.

(51) Int. Cl. A61K 36/21; A23L 2/52; A23L 33/155; A61K 31/593

(52) U.S. Cl. CPC A61K 36/21; A61K 31/593; A23L 2/52; A23L 33/155; A23V 2002/00

(57) ABSTRACT

Embodiments of the inventions described herein provide, among other things, enriched Amaranthus blitoides compositions, such as those enriched with Vitamin D and to the use thereof to alleviate symptoms or discomforts associated with inflammation, inflammatory disorders, Benign Prostatic Hyperplasia (BPH), other inflammation related problems, infections of the genitourinary tract, hemorrhoidal diseases and the same, and to use of the same to treat these and other disorders and diseases.

### FIELD OF THE INVENTION

Embodiments of inventions herein described relate to enriched Amaranthus blitoides compositions, including those enriched with Vitamin D, extracts and teas prepared from the same, and uses thereof for the relief of discomforts and disorders, such as those due to or associated with inflammation, for example symptoms commonly due to Benign Prostatic Hyperplasia (BPH), prostititous of the female prostate, infections of the genitourinary tract, and hemorrhoidal diseases, to name a few, and to treatments thereof.

### GOVERNMENT FUNDING

No government funds were involved in making the invention herein described.

### I

Amaranthus blitoides is a common member of the pigweed family. It occurs in much of the United States and Canada, as well as in Europe and other areas of the world. It has been used for some time, particularly as tea, for relief from a variety of discomforts and symptoms, such as those caused by or associated with inflammation and inflammatory processes, such as symptoms of Benign Prostatic Hyperplasia (BPH), and those often associated with infections of the genitourinary tract or hemorrhoidal diseases. Various preparations of A. blitoides, primarily dried preparations for making teas, are available commercially in Europe, where they enjoy some popularity. Such commercial offerings are not as common in the US; although, they are freely available. Its widespread use in Europe, particularly for relief from BPH, is popular confirmation that it is effective in relieving discomfort; something that has been confirmed by more systematic studies. Medical uses of the plant also are known, there are studies reporting on its treatment efficacies. For example see the following review: Bogdan Boskovic, “Pharmacological-Toxicological-Clinical Expert Report for Amer Tea”; National Center for Toxic Control, Belgrade, Serbia (2005) which is herein incorporated in its entirety in parts pertinent to the use of Amaranthus to treat disorder or disease, or medicinal purposes. In addition, a German patent has been granted the use of Amaranthus Blitoides to treat BPH (DE502004009359C0, Verwendung von Teilen Oder Eines Extrakts von Amaranthus Blitoides counterpart of EP1641477B1 for Use of Parts or Extract of Amaranthus Blitoides and of PCT/EP2004/006235, International Publication Number WP04112812A on Use of Parts or Extract of Amaranthus Blitoides and see the related disclosure DE10332281A1 on Medicaments for Treating Inflammatory Diseases, Especially Hemorrhoids, Herpes or Urinary Tract or Prostate Inflammation, Comprises Parts or Extracts of the Plant Amaranthus Blitoides, each of which is herein incorporated in its entirely in parts pertinent to treating diseases and disorders with Amaranthus.

Unfortunately, the palliative effect of these preparations is incomplete, as is their curative power. All too often their use does not relieve symptoms and discomfort or underlying disease as fully as users and care providers desire. Naturally, therefore, it would be beneficial to have improved compositions of Amaranthus blitoides (and other members of the pigweed family that provide similar relief), as well as more effective teas and other administrative forms for the relief of discomforts and symptoms, such as those due to or associated with inflammation, for example symptoms commonly due to Benign Prostatic Hyperplasia (BPH), prostititous, including prostititous of the female prostate, infections of the genitourinary tract, and hemorrhoidal diseases, and for treatment of these diseases as well.

### II

Illustrative, non-limiting embodiments of inventions described herein are set forth illustratively in the following numbered paragraphs in this section. The phrase “In accordance with any of the foregoing or the following” used in these numbered paragraphs refers to the other numbered paragraphs in the section. In these paragraphs the phrase explicitly means that the illustrative embodiments described herein include the subject matter set forth in each numbered paragraph by itself and in any combination with the subject matter of any one or more of the other numbered paragraphs. It is explicitly applicant's purpose in setting forth these numbered paragraphs to describe various embodiments by the paragraphs taken alone and in any combination with one another, including, where any paragraph lists alternatives, any one or more of the alternatives in combination with any one or more of the alternatives listed in any one or more of the other paragraphs. Embodiments are thus commodiously disclosed as including the subject matter of each paragraph alone, including alternatives, and in any combination with the subject matter of any other paragraphs, including any one or more of alternatives therein set forth. Applicant specifically reserves the right to claim any of the subject matter set out in these paragraphs, taking the paragraphs alone or in any combination, including any one or more alternatives listed in any one or more of the paragraphs taken alone or in any combination with one or more of any other paragraphs.

1. A composition comprising Vitamin D and Amaranthus blitoides in a ratio of any of about or 1, 2, 3, 4. 5. 10, 15, 20, 25, 30, 40, 50, 60, 75, 100, 125, 150, 175, 200, 225, 250, 275, 300, 325, 350 IU of Vitamin D per gram equivalent of Amaranthus blitoides, or the same in accordance with any one or more of the following, in whole or in any part or parts.

2. A composition according to any of the foregoing or the following wherein the ratio of Vitamin D or Vitamin D derivative to Amaranthus blitoides is in the range of any of 1-2,000, 2-2,000, 5-1,000, 5-500, 10-1,000, 10-500, 20-1,000, 20-500, 50-1,000, 50-500, 50-250, 50-125, 25-250, 25-750, 75-750, 75-500, 75-225, 0.1-100, 0.5-50, 0.25-25, 1-25, 1-15, 1-10 IU of Vitamin D per gram equivalent of Amaranthus blitoides.

3. A composition according to any of the foregoing or the following wherein the Amaranthus blitoides is Amaranthus blitoides S. Watson.

4. A composition according to any of the foregoing or the following wherein Vitamin D is substantially only any one or more of Vitamin D₁, Vitamin D₂, Vitamin D₃, Vitamin D₄, Vitamin D₅ or one or more derivatives thereof.

5. A composition according to any of the foregoing or the following wherein the Amaranthus blitoides is cut, minced, crushed, powdered, pulverized or a mixture of any one or more thereof in any proportion thereof.

6. A composition according to any of the foregoing or the following wherein the Amaranthus blitoides is in the form of whole leaves, minced leaves, crushed leaves, powdered leaves, pulverized leaves or a mixture of any one or more in any proportion thereof.

7. A composition according to any of the foregoing or the following, further comprising one or more additional substances suitable for human consumption.

8. A composition according to any of the foregoing or the following, further comprising any one or more of the following in any number and combination: fillers, preservatives, colorants, sweeteners, or flavorings suitable for human consumption.

9. A composition according to any of the foregoing or the following, further comprising additional vitamins, minerals or other nutrients suitable for human consumption.

10. An extract of any of the foregoing or the following compositions.

11. An aqueous extract of any of the foregoing or the following compositions, said extract being suitable for human consumption and further comprising any one or more fillers, preservatives, colorants, sweeteners, or flavorings.

12. An aqueous extract of any of the foregoing or the following compositions, said extract being suitable for human consumption and further comprising any one or more additional vitamins, minerals or other nutrients.

13. A beverage suitable for human consumption comprising or made from any one or more of the foregoing or the following compositions or extracts.

14. An energy drink, tea, lemonade, juice, juice drink, soft drink, water or flavored water drink or nutritional food drink comprising or made from any one or more of the foregoing or following compositions or extracts.

15. A tea suitable for human consumption comprising or made from any one or more of the foregoing or the following compositions or extracts.

16. A sport drink suitable for human consumption comprising or made from any one or more of the foregoing or the following compositions or extracts.

17. A concentrated extract comprising or made from any of the foregoing or the following compositions, wherein said extract is suitable for making a tea or a sport drink for human consumption.

18. A composition according to any of the foregoing or the following disposed in a capsule, caplet, tablet, tab or other solid formulation suitable for human use.

19. A water permeable container having disposed therein a composition according to any of the foregoing or the following.

20. A water permeable container having disposed therein a composition according to any of the foregoing or the following, said container being made of filter paper, nylon, silk or Soilon.

21. A water permeable container having disposed therein a composition according to any of the foregoing or the following in an amount of about 0.1, 0.2, 0.5, 1.0, 2.0, 3.0, 4.0, 5.0, 7.0, 10, 15, 20, 25, 50, 100, 150, 200, 250 or 500 grams.

22. A water permeable container having disposed therein a composition according to any of the foregoing or the following in the range of any of 0.1-100, 0.5-500, 0.5-25, 1-5, 1-10, 1-10,000, 2-4, 2-20, 2-200, 15-150, 25-750 or 50-5,000 grams.

23. A capsule, caplet, tablet, tab or other solid formulation suitable for human use comprising a composition according to any of the foregoing or the following.

24. A capsule, caplet, tablet, tab or other solid formulation suitable for human use comprising any of about 0.1, 0.2, 0.5, 1.0, 2.0, 3.0, 4.0, 5.0, 7.0, 10, 15, 20, 25, 50, 100, 150, 200, 250 or 500 grams of a composition according to any of the foregoing or the following.

25. A capsule, caplet, tablet, tab or other solid formulation suitable for human use comprising any of about 0.1 to 0.5, 0.2 to 1.0, 0.5 to 2.0, 1.0 to 3.0, 2.0 to 4.0, 3.0 to 5.0, 2.5 to 7.5 5.0 to 10, 7.5 to 15, 10 to 20, 15 to 30, 25 to 50, 30 to 60, 50 to 100, 75 to 125, 100 to 150, 125 to 175, 150 to 200, 175 to 250 200 to 300, 250 to 500 or more grams of a composition according to any of the foregoing or the following.

26. A method for treating one or more symptoms or discomforts due to any one or more of inflammation, arthritis, Benign Prostatic Hyperplasia (BPH), prostititous, including prostititous of the female prostate, an infection of the genitourinary tract, and/or a hemorrhoidal disease, comprising administering to a patient in need thereof a composition or extract in accordance with any of the foregoing or the following.

27. A method for treating one or more symptoms or discomforts due to an immune-related disorder, comprising administering a composition in accordance with any of the foregoing or the following.

28. A method for treating inflammation, Benign Prostatic Hyperplasia (BPH), prostititous, including prostititous of the female prostate, infections of the genitourinary tract, and/or a hemorrhoidal diseases, comprising administering a composition or extract in accordance with any of the foregoing or the following.

29. A method for treating an immune-related disorder, comprising administering a composition in accordance with any of the foregoing or the following.

### III

IU (International Unit) is used herein in relationship to the various forms of Vitamin D and derivatives thereto to denote International Unit and, as described below, is used herein to denote 65 picomoles Vitamin D₃ and equivalents thereof.

For Vitamin D₃, one IU is 0.025 micrograms of pure 3β,5Z,7E)-9,10-secocholesta-5,7,10(19)-trien-3-ol (the IUPAC name for Vitamin D₃, which also is called cholecalciferol and activated 7-dehydrocholesterol). Since the molecular weight of Vitamin D₃ is 384.64 g/mol, one IU of Vitamin D₃ is also 65 picomoles of pure 3β,5Z,7E)-9,10-secocholesta-5,7,10(19)-trien-3-ol. It will be appreciated that the mass of 65 picomoles of a substance will depend on its molecular weight and is different for substances of different molecular weights. Likewise, the “activity” in a given assay, such as an assay of Vitamin D activity, of a given amount of a substance, such as 65 picomoles, will depend on the specific activity of the substance, and will depend on the intrinsic specific activity of the substance if the substance is in completely pure and fully active form. For purposes herein, should there be ambiguity or doubt about its meaning, one IU of a Vitamin D substance means 65 picomoles of the substance. Milk in the United States is fortified with 100 IU Vitamin D per cup; that is, approximately 2.5 microgram of Vitamin D per cup of milk.

A gram equivalent of Amaranthus blitoides, as that term is used herein, is one gram of dried leaves of Amaranthus blitoides, or the amount of any extract, derivative, infusion, and the like derived or made from one gram of dried leaves. For instance, one gram equivalent of a liquid infusion of Amaranthus blitoides would be the amount of the infusion obtained per gram of dried leaves. For an infusion made by steeping 25 grams of dried leaves in 250 ml of water, 10 ml would contain a gram equivalent of the Amaranthus. If the same infusion was concentrated from 250 ml to 50 ml, each 2 ml would contain a gram equivalent of the Amaranthus.

The term container is used herein in this respect to mean much the same thing as pouch or bag or the like, particularly in reference the use of a water permeable barrier suitable for holding the compositions herein disclosed while at the same time allowing them to come in contact with liquid, infuse there into, and then be conveniently removed there from, in much the same manner as a tea bag serves as a container for tea allowing the tea to be put in contact with hot water, infuse it, and then be conveniently removed from the water.

### IV

Herein described are supplemented Amaranthus blitoides compositions useful for relief of symptoms and discomfort associated with inflammation, such as that caused by inflammatory disorders, Benign Prostate Hyperplasia, prostititous, including prostititous of the female prostate, infections of the genitourinary tract, hemorrhoidal diseases, and inflammatory disorders to name a few. The supplemented compositions as disclosed herein also are useful to treat the same, and methods are herein disclosed for using them to that end.

### Amaranthus blitoides

Amaranthus blitoides is an edible herb of the family Amaranthaceae. It is often referred to as Prostrate amaranth, Matweed, and Mat. It is has been used as a food for both humans and domestic animals for centuries—if not longer—without notable side effects. It is particularly popular in many countries in Europe including, for instance, Germany, France, Italy, Croatia, Serbia and Montenegro, and it is commonly available in various forms in health food and organic food shops in the United States. It is made into flour and used to make breads, pastas and other baked goods. There is no gluten in Amaranthus.

It is, like all the Amaranthaceae, monoecious. It has separate male (staminate) and female flowers (pistillate). Male flowers have 4-5 sepals, 3 stamens, and no petals. Female flowers have 4-5 sepals, an ovary with 3 styles, and no petals. The sepals of both male and female flowers are oblong lanceolate shaped and approximately ⅛″ in length.

It is believed to have originated in central or eastern United States. Today, it is found in the wild throughout much of temperate North America, South America and Eurasia. Typically, it grows in moist fertile soil in sunny areas. Plants generally grow to 0.6 m to 1 m. They are summer annuals and wind pollinated. Flowers have three stigmas in the form of axillary clusters. The cuspidate floral bracts are as long as the flowers themselves. Each flower gives rise to a capsule. Each capsule releases one seed. The seeds are edible round, flattened discs, shiny dark brown to black in appearance, about 1.1-1.7 mm in diameter. The plant and its cultivation, harvesting, drying and preparation are well known, and it is available commercially in several forms.

Amaranthus blitoides, on a weight percent basis, contains more protein than milk, three times as much fiber and five times as much iron as wheat, and high levels of vitamins A and C, calcium, potassium, and phosphorous. The seeds contain fairly high levels of lysine, which is lacking in other grains. The plant also contains many macro and micro nutrients including, but not limited to vitamin C, carotene, starch, proteins, fatty oils, sugar, cellulose, flavonoids, tannins, betanine, saponins, glycoproteins, β-sitosterol, TRF, Fe, Mn, Cu, Co, K, Zn, and Na, and it is a rich source of camesterol and stigmasterol. It has long been known for its analgesic, antiphologistic, and spasmolytic properties, no doubt owing to these and other ingrediants. For instance, flavonoids bind free radicals and have anti-inflammatory, antimicrobial, and antiviral effects. Saponins have been shown to have anti-inflammatory effects. The proteins and glycoproteins in the plant are reputed to have antimicrobial and antiedematous effects. B-sitosterol is a phytosterol that may exercise anti-inflammatory effects by interfering with prostaglandin metabolism, and it has been reported to have anti-benign prostatic hyperplasia (BPH) activity. Two other phytosterols found in Amaranthus, camesterol and stigmasterol, have been reported to improve cholesterol levels and to be of some help to those suffering from peripheral artery and coronary artery disease.

In an embodiment Amaranthus blitoides leaves are used. In an embodiment the leaves are dried. In an embodiment the leaves are any one or more of minced, chopped, crushed, pulverized, powdered, or broken up in other ways. In an embodiment that leaves can be prepared for use in or for making any one or more of infusions such as teas, tinctures, extracts such as concentrated extracts for making beverages including, for instance, health, nutritional supplement and sport drinks, and for formulation into, among others, solid dispersions, liquid dispersions, capsule, tablet or other forms.

### Vitamin D

Vitamin D is an essential nutrient in human physiology. The two forms primarily of importance to human nutrition are Vitamin D₂ (ergocalciferol) and Vitamin D₃ (cholecalciferol). Other forms of vitamin D include Vitamin D₁ (a 1:1 mixture of ergocalciferol and lumisterol), Vitamin D₄ (22-dihydroergocalciferol) and Vitamin D₅ (sitocalciferol). Vitamins D₂ and D₃ both are precursors to physiologically active compounds. Both are activated by two hydroxyl addition reactions. The first generally occurs in the liver. The second occurs either in kidney, resulting in hormone activity, or in cells of the immune system, in which case the activated vitamin exhibits cytokine activity.

The activated form of Vitamin D binds to vitamin D receptors. The Vitamin D-Vitamin D Receptor complexes (vD-vDR) form heterodimers with retinoid X receptors (RXR). The heterodimers bind to vitamin D-responsive elements in the regulatory regions of vitamin D sensitive genes. Binding of the heterodimers to the genes effectuates changes in transcription. The transcriptional effects are mediated not only by heterodimer binding but also by a panoply of co-activators, including CBP/SRC coactivator complex, Vitamin D receptor interacting protein complex (DRIP), RAS activated Ets transcription factor, SWI/SNF complex and members of the CCAAT enhancer binding protein family Vitamin D-responsive genes generally encode proteins involved in calcium and phosphate transport and its regulation, particularly transport proteins, of which TRPV6 and calbindin are particularly notable.

The molecular biology mirrors the main physiological activity of Vitamin D in regulating proper levels of calcium and phosphorous, particularly through their absorption in the intestine. These activities explain well known pathophysiologiocal effect of Vitamin D deficiency: inadequate mineralization and—via mobilization of calcium, in particular from bone—demineralization, causing rickets in children and ostomalacia and hyperparathyroidism in adults.

Vitamin D deficiencies are largely prevented in the economically developed countries of the world by government mandated supplementation of certain foods with Vitamin D. In the US, for instance, this is accomplished mainly by “fortifying” milk with 100 IU of Vitamin D per cup. However, there is evidence suggesting that many Americans, particularly women, still suffer from Vitamin D deficiency and that it would be very beneficial for them to have additional Vitamin D in their diets. (See Christakos et al. (2003), J. Cellular Biochem. 88: 695-705; Christakos et al. (2006), Ann. N.Y. Acad. Sci. 1068: 194-203; Christakos et al. (2007), Ann, N.Y. Acad. Sci. 1116: 340-348 and National Report on Biochemical Indicators of Diet and Nutrition in the U.S. Population (1999-2002), Fat Soluble Vitamins & Nutrients: Vitamin D; pgs 61-69, for example). Moreover, there are populations that do not tolerate milk and would benefit from an additional supplemented dietary source. Vitamin D enriched Amaranthus blitoides provides such an alternative source of the Vitamin D.

Vitamin D also has been shown to play a role in immune regulation and to have activity against certain types of cancer. It has been shown to inhibit proliferation and promote differentiation of several tumor cell types, including prostate cancer cell lines, and primary prostate tumor lines, and to decrease invasion of prostate cancer cells in vitro (Sung et al. (200x): 1,25-Dihydroxyvitamin D3 decreases human prostate cancer cell adhesion and migration” Department of medicine, Division of Endocrinology, Gerontology and Metabolism, Stanford U., Sch. Med, Web). In addition, Vitamin D has been shown to induce decreased expression of α6 and β4 integrins, which are associated with increased migration and invasion of prostate cancer cells in vitro (Sung et al. (2000) as above and Sung et al. (2000), Molec. Cellular Endocrin. 164: 133-143. Furthermore, the Vitamin D analog, BXL-628 has been shown to arrest prostate growth and reduce prostate volume in Phase IIa clinical trials (Colli et al. (2006), European Urology 49: 82-86). See also WO 2005/027923: Use of Vitamin D3 Analogs to Treat Prostate Hyperplasia.

### Sources of Vitamin D Compounds

Vitamin D is available commercially in a variety of forms suitable for human consumption and for use in accordance with various embodiments of the inventions herein disclosed. In this regard it may be obtained as any one or a mixture of any combination of Vitamin D vitamers including Vitamin D₁, Vitamin D₂, Vitamin D₃, Vitamin D₄, and/or Vitamin D₅.

Vitamin D₃, for example, is available commercially in a variety of soluble formulations suitable for human consumption that can be used in embodiments described herein. Falcon Trading International, offers Vitamin D3 Chlecalciferol; 40,300,000 IU/gram by HPLC, Grade USP25-BP2000, soluble white crystalline powder, Melting Point 82-87° C.; Absorption 0.46-0.50; Optical Rotation +105-+112°; Specific activity 38,800,000-41,200,000 IU/g (97.0-103.0% purity); 7-Dehydrocholesterol <0.2%.

LycoRed, another company, provides a variety of Vitamin D formulations suitable for human consumption. One formulation is a powdered form in which the vitamin D is stabilized in a beadlet matrix suitable for use in dietary supplements, and for tablet and hard-shell capsule applications. Vitamin D₂ and D₃ formulations of this type are offered, in the following specific activities: Vitamin D₂ 100,000 and 850,000 IU/g; Vitamin D₃ 100,000, 500,000 and 1,000,000 IU/g. Microencapsulated powders specially formulated for food and beverage fortification also are available, including Vitamin D₂ CapsuDar® D2 100E (100,000 IU/g) and Vitamin D₃ CapsuDar® D3 100E (100,000 IU/g). Cold water dispersible powder formulations suitable for food and beverage fortification also are available, including Vitamin D₂ CapsuDar® D2 100 CWD (100,000 IU/g) and Vitamin D₃ CapsuDar® D3 100 CWD (100,000 IU/g). And oil suspensions suitable for use in soft-gel capsules and oil based applications are available from this company, including formulations of Vitamin D2 at 1,000,000 IU/g and Vitamin D3 at 1,000,000 IU/g.

These and other such formulations may be used in accordance with various embodiments of the inventions herein described.

### Metabolism and Dosage of Vitamin D

Healthy adults tolerate a wide range of Vitamin D doses; but, it can be toxic in excessive amounts. Toxicity has been seen in healthy adults at dosages above about 1250 μg/day. Daily ingestion of 375 μg/kg (about 15 mg for a 90 lb person) of Vitamin D/day has been shown to produce acute toxicity in adults within two weeks. Toxicity has been observed in adults following long term daily ingestion of 25 μg/kg of Vitamin D (about 1 mg for a 90 lb person). Certain medical conditions increase sensitivity to vitamin D and, in extreme cases, even mildly elevated Vitamin D levels may result in adult hypercalcaemia. Hypercalcaemia in pregnant women has been associated with congenital mental retardation and facial deformities. See, for instance, Remington: The Science and Practice of Pharmacy 21^{st} Ed., P. Beringer, pps 1696-1697, Lippincott, Williams & Wilkins, Baltimore, Md. (2006).

### Amaranthus blitoides—Vitamin D Compositions

Certain embodiments provide compositions comprising Amaranthus blitoides and Vitamin D. In certain embodiments the Amaranthus blitoides and Vitamin D are obtained and combined in conventional ways. In certain embodiments the Amaranthus blitoides and Vitamin D are obtained form commercial suppliers. In certain embodiments the Amaranthus blitoides and Vitamin D are mixed together by conventional means. In certain embodiments Vitamin D is formulated for water solubility and the composition comprising Amaranthus blitoides and Vitamin D is or is suitable for making a beverage, such as a beverage for human consumption. In certain embodiments, the composition comprising Amaranthus blitoides and Vitamin D is or is suitable for making a tea, such as a tea suitable for human consumption. In certain embodiments the composition comprising Amaranthus blitoides and Vitamin D is disposed in a container for making an infusion or tea, such as a tea bag.

In certain embodiments the Amaranthus blitoides is Amaranthus blitoides S. Watson.

In certain embodiments the Vitamin D is one or more of Vitamin D₁, Vitamin D₂, Vitamin D₃, Vitamin D₄, Vitamin D₅ or a derivative thereof effective to provide the benefits of Vitamin D to a subject.

In certain embodiments the Amaranthus blitoides is cut, minced, crushed, powdered or pulverized or a mixture of any one or more thereof.

In certain embodiments the Amaranthus blitoides is in the form of whole leaves, minced leaves, crushed leaves, powdered leaves, pulverized leaves or a mixture of any one or more of the foregoing in any proportion thereof.

In certain embodiment the ratio of Vitamin D to Amaranthus blitoides is any of about 1, 2, 3, 4. 5. 10, 15, 20, 25, 30, 40, 50, 60, 75, 100, 125, 150, 175, 200, 225, 250, 275, 300, 325, 350 IU of Vitamin D per gram equivalent of Amaranthus blitoides.

In certain embodiment the ratio of Vitamin D to Amaranthus blitoides is in the range of any of 1-2,000, 2-2,000, 5-1,000, 5-500, 10-1,000, 10-500, 20-1,000, 20-500, 50-1,000, 50-500, 50-250, 50-125, 25-250, 25-750, 75-750, 75-500, 75-225, 0.1-100, 0.5-50, 0.25-25, 1-25, 1-15, 1-10 IU of Vitamin D per gram equivalent of Amaranthus blitoides.

In certain embodiments the composition further comprises substances, such as additional substances suitable for human consumption.

In certain embodiments the composition further comprises any one or more of the following in any number and combination: fillers, preservatives, colorants, sweeteners, or flavorings suitable for human consumption.

In certain embodiments the composition further comprises any one or more of additional metals, minerals, organics, vitamins, amino acids and other substances or nutrients, such as those suitable for human consumption. In certain embodiments the composition comprises, any one or more of the following: zinc, copper, iodine, chromium, iron, molybdenum, selenium, silicon, boron, vanadium, germanium, potassium, calcium and/or magnesium; polyphenolics, sterols, such as sitosterol, and/or free fatty acids; Vitamin C, Vitamin E, Vitamin B-6 and/or other vitamins; lysine, glycine and/or tyrosine; saw palmetto, lycopene, quercetin, Pyseum africanum, stinging netttles, pumpkin seed oil, sasparilla, ginseng, Bubella broom, milk thistle, kelp, Uva ursi extract, flower pollen, bosvellio, flaxseed, bees wax, calcium D glycan, and/or glycinine, such as to all of the foregoing those suitable for human consumption in amounts beneficial to human health.

In certain embodiments the composition is an extract, tincture or other liquid made from Amaranthus blitoides supplemented with Vitamin D in the ratios set forth herein, and optionally further supplemented with any one or combination of ingredient set forth above, such as but not limited to thickeners, fillers, preservatives, colorants, sweeteners, or flavorings. In embodiments the extract is a concentrated extract for making a tea, sport drink or other beverage, such as by dilution.

In embodiments the composition is a beverage, such as a beverage suitable for human consumption, such as, among others a tea, sport drink, energy drink, lemonade, juice or juice drink, soft drink, water or flavored water drink or nutritional food drink with the concentration of Amaranthus blitoides, Vitamin D and optionally other ingredients set forth above and elsewhere herein.

In embodiments the composition is disposed in a capsule, caplet, tablet, tab or other solid formulation suitable for human use. In embodiments the composition is disposed in water permeable container, such as a container for brewing tea. In embodiments the composition is disposed in a container comprising any one or more of the following: filter paper, nylon, silk or Soilon.

Embodiments provide a water permeable container having disposed therein a composition according to any of the foregoing or the following in an amount of about 0.1, 0.2, 0.5, 1.0, 2.0, 3.0, 4.0, 5.0, 7.0, 10, 15, 20, 25, 50, 100, 150, 200, 250 or 500 grams.

A water permeable container having disposed therein a composition according to any of the foregoing or the following in the range of any of 0.1-100, 0.5-500, 0.5-25, 1-5, 1-10, 1-10,000, 2-4, 2-20, 2-200, 15-150, 25-750 or 50-5,000 grams.

Embodiments provide a caplet, tablet, tab or other solid formulation, such as those suitable for human use, comprising any of about 0.1, 0.2, 0.5, 1.0, 2.0, 3.0, 4.0, 5.0, 7.0, 10, 15, 20, 25, 50, 100, 150, 200, 250 or 500 grams of a composition as described above and elsewhere herein.

Embodiments provide a capsule, caplet, tablet, tab or other solid formulation suitable for human use comprising any of about 0.1 to 0.5, 0.2 to 1.0, 0.5 to 2.0, 1.0 to 3.0, 2.0 to 4.0, 3.0 to 5.0, 2.5 to 7.5 5.0 to 10, 7.5 to 15, 10 to 20, 15 to 30, 25 to 50, 30 to 60, 50 to 100, 75 to 125, 100 to 150, 125 to 175, 150 to 200, 175 to 250 200 to 300, 250 to 500 or more grams of a composition as described above and elsewhere herein.

### Methods of Treatment

Compositions comprising Amaranthus blitoides and Vitamin D as described herein may be used to reduce symptoms and/or discomfort associated with inflammation, arthritis, Benign Prostatic Hyperplasia (BPH), prostititous, including prostititous of the female prostate, an infection of the genitourinary tract, and/or a hemorrhoidal disease. In embodiments the treatment does not cause unwanted sexual dysfunction.

Compositions comprising Amaranthus blitoides and Vitamin D as described herein may be used to treat inflammation, arthritis, Benign Prostatic Hyperplasia (BPH), prostititous, including prostititous of the female prostate, an infection of the genitourinary tract, and/or a hemorrhoidal disease. In embodiments treatment does not cause unwanted sexual dysfunction.

Embodiments provide methods for treating one or more symptoms and/or discomforts due to any one or more of inflammation, arthritis, Benign Prostatic Hyperplasia (BPH), prostititous, including prostititous of the female prostate, an infection of the genitourinary tract, and/or a hemorrhoidal disease, comprising administering to a subject in need thereof an Amaranthus blitoides—Vitamin D composition as described above and elsewhere herein.

Embodiments provide methods for treating one or more symptoms and/or discomforts due to an immune-related disorder, comprising administering to a subject in need thereof an Amaranthus blitoides—Vitamin D composition as described above and elsewhere herein.

Embodiments provide methods for treating any one or more of inflammation, Benign Prostatic Hyperplasia (BPH), prostititous, including prostititous of the female prostate, infections of the genitourinary tract, and/or a hemorrhoidal diseases, comprising administering an Amaranthus blitoides—Vitamin D composition as described above and elsewhere herein.

Embodiments provide methods for treating an immune-related disorder, comprising administering an Amaranthus blitoides—Vitamin D composition as described above and elsewhere herein.

### V

The following Examples are provided by way of illustration only and are in no way limiting of the inventions herein described or their embodiments. A more complete understanding of the inventions herein described is to be had only by reading the present application in its entirety as it would be understood by those persons skilled in the arts to which it pertains.

### EXAMPLE 1

### Preparation of Amaranthus blitoides

Amaranthus blitoides leaves are removed from the plant and allowed to dry. The leaves are then crushed or chopped.

### EXAMPLE 2

### Preparation of Amaranthus blitoides—Vitamin D Compositions

Two kilograms of crushed dried leaves of Amaranthus blitoides mixed with one gram of CapsuDar® D3 100E (100,000 IU/g), a microencapsulated powder formulation of Vitamin D3 for foods, providing an enriched Amaranthus blitoides—Vitamin D composition with 100 IU (1 milligram) Vitamin D/2 grams Amaranthus blitoides.

### EXAMPLE 3

### Preparation of Tea Bags of Enriched Amaranthus blitoides

Two grams each of the composition set out in Examiner 2 are introduced into tea bags in accordance with standard practices for making tea bags use in foods for human consumption. Each tea bag thus produced contains approximately 2 grams dried leaves of Amaranthus blitoides and 1 milligram of microencapsulated Vitamin D preparation. Fully extracted the Vitamin D in each tea bag provides 100 IU, about the same as a cup of Vitamin D fortified milk sold in the United States.

### EXAMPLE 4

### Amaranthus blitoides—Vitamin D Tea

A tea bag containing Amaranthus blitoides enriched with Vitamin D according to Example 3 is steeped in one cup of boiled water for approximately 5 minutes. The resulting solution is enriched tea in accordance an embodiment of the invention herein described.

### EXAMPLE 5

### Concentrate of Sport Drink

Amaranthus blitoides tea is brewed as described in Example 4. The tea is concentrated 100 fold by vacuum evaporation or freeze evaporation (lyophilization). The resulting concentrate is added to a sport drink formulation in a ratio of 1:99 to provide Amaranthus blitoides—Vitamin D enriched sport drink. Alternatively the concentrate is made from Amaranthus blitoides prior to enrichment with Vitamin D, and Vitamin D is added in the appropriate amount to the concentrate, to the sport drink formulation or to the sport drink.

### EXAMPLE 6

### Capsules

For the production of a capsule, dried pulverized leaves of Amaranthus blitoides is mixed with Vitamin D powder and put into a pharmaceutically acceptable capsule.

### EXAMPLE 7

### Treatment of Symptoms Associated with Inflammation

A cup of tea prepared as described in Example 4 is ingested twice each day for at least two weeks by a subject suffering from symptoms associated with inflammation. By the end of the two weeks the subject experiences at least partial relief from the symptoms.

### EXAMPLE 8

### Treatment of Symptoms Associated with BPH

A cup of tea prepared as described in Example 4 is ingested twice each day for at least two weeks by a subject suffering from symptoms associated with BPH. By the end of two weeks the subject experiences at least partial relief from the symptoms of BPH.

### EXAMPLE 8

### Treatment of Symptoms Associated with Inflammation

One capsule of A. blitoides—Vitamin D as described in Example 6 is ingested twice each day for at least two weeks by a subject suffering from symptoms associated with inflammation. By the end of the two weeks the subject experiences at least partial relief from the symptoms.

### EXAMPLE 9

### Treatment of Symptoms Associated with BPH

One capsule of A. blitoides—Vitamin D as described in Example 6 is ingested twice each day for at least two weeks by a subject suffering from symptoms associated with BPH. By the end of two weeks the subject experiences at least partial relief from the symptoms of BPH.

What is claimed is:

1. A composition comprising Vitamin D or a Vitamin D derivative and Amaranthus blitoides, wherein the ratio of the Vitamin D or Vitamin D derivative to the Amaranthus blitoides is 2-2,000.

2. A composition according to claim 1, wherein the Amaranthus blitoides is Amaranthus blitoides S. Watson.

3. A composition according to claim 1, wherein the Vitamin D or Vitamin D derivative is Vitamin D₃.

4. A composition according to claim 2, wherein the Vitamin D or Vitamin D derivative is Vitamin D₃.

5. A beverage suitable for human consumption comprising or made from a composition according to claim 1.

6. A tea according to claim 5.

7. A sport drink according to claim 5.

8. An energy drink according to claim 5.

9. A water permeable container having disposed therein a composition according to claim 1.

10. A water permeable container according to claim 8 having disposed therein 0.1-500 grams of a composition according to claim 1.

11. A solid formulation suitable for human use comprising a composition according to claim 1.

12. A solid formulation according to claim 11, comprising 0.5-25 grams of a composition according to claim 1.

13. A method for treating a symptom or discomfort due to inflammation, arthritis, Benign Prostatic Hyperplasia, prostititous, including prostititous of the female prostate, an infection of the genitourinary tract or a hemorrhoidal disease, comprising administering to subject in need thereof a composition comprising Vitamin D or a Vitamin D derivative and Amaranthus blitoides, wherein the ratio of the Vitamin D or Vitamin D derivative to the Amaranthus blitoides is 2-2,000.

14. A method according to claim 13, for treating a symptom or discomfort due to Benign Prostatic Hyperplasia.

15. A method according to claim 13, for treating a symptom or discomfort due to prostititous of the female prostate

16. A method according to claim 13, for treating a symptom or discomfort due to inflammation.

17. A method for treating inflammation, arthritis, Benign Prostatic Hyperplasia, prostititous, including prostititous of the female prostate, an infection of the genitourinary tract or a hemorrhoidal disease, comprising administering to subject in need thereof a composition comprising Vitamin D or a Vitamin D derivative and Amaranthus blitoides, wherein the ratio of the Vitamin D or Vitamin D derivative to the Amaranthus blitoides is 2-2,000.

18. A method according to claim 17, for treating Benign Prostatic Hyperplasia.

19. A method according to claim 17, for treating prostititous of the female prostate.

20. A method according to claim 17, for treating inflammation.
